(19) United States
(12) Patent Application Publication
(10) Pub. No.: US 2022/0031736 A1
(43) Pub. Date: Feb. 3, 2022

(54) USE OF ANIONIC CLAY IN PREPARING LEAD-REMOVING MEDICINE

(71) Applicant: Dejun MA, BEIJING (CN)

(72) Inventors: Dejun MA, BEIJING (CN); Xinqi CHEN, BEIJING (CN); Feng HAN, BEIJING (CN); Renjie YAN, BEIJING (CN)

(21) Appl. No.: 17/502,105

(22) Filed: Oct. 15, 2021

Related U.S. Application Data
(63) Continuation of application No. PCT/CN2020/084342, filed on Apr. 11, 2020.

(30) Foreign Application Priority Data
Apr. 16, 2019 (CN) 201910303230.8

(51) Int. Cl. A61K 33/08; A61K 45/06; A61P 39/02

(52) U.S. Cl. CPC A61K 33/08; A61P 39/02; A61K 45/06

(57) ABSTRACT

Disclosed is a new medicinal use of anionic clay, and particularly to a use of anionic clay in preparing a lead-removing medicine. The described medicinal use of the anionic clay is capable of effectively preventing and treating lead poisoning and excessive lead, has significant lead-removing effects and little side effects, and has positive clinical significance.

### CROSS-REFERENCE TO RELATED APPLICATIONS

This application is a continuation of International Patent Application No PCT/CN2020/084342 with a filing date of Apr. 11, 2020, designating the United States, now pending, and further claims priority to Chinese Patent Application No. 201910303230.8 with a filing date of Apr. 16, 2019. The content of the aforementioned applications, including any intervening amendments thereto, are incorporated herein by reference.

### TECHNICAL FIELD

The present invention belongs to the field of medicine, and particularly relates to a use of anionic clay in preparing a lead-removing medicine.

### BACKGROUND

In recent years, with the rapid growth of population, the rapid development of industry, and the abuse of pesticides and fertilizers, a large number of harmful heavy metals have been discharged one after another, resulting in environmental pollution. The accumulation, migration and transformation of these harmful substances in the environment, through food chains, have caused harm to animal and human health. The influence of heavy metal lead on the growth, development and health of children and adolescents is the focus of attention in the pharmaceutical industry.

At present, the research on medicines for clinical treatment of lead poisoning is progressing slowly at home and abroad. At present, most of the effective therapeutic agents are complexing agents, which form a nontoxic and easily excreted lead complex with lead ions and excrete the lead complex through the kidney. These complexing agents may be divided into aminocarboxylic acid compounds and sulfhydryl compounds according to structures. The sulfhydryl compounds include penicillamine and dimercaptosuccinic acid. At present, the clinically-used medicines for promoting excretion generally have shortcomings such as limited ability to promote excretion, great toxic and side effects, poor water solubility, foul smell, being unfavorable for oral administration, poor effects on patients with chronic kidney disease, or the like, which limit these medicines to play a role in the treatment of heavy metal lead excretion. At present, the lead poisoning of Level III and below in the international diagnostic standards for lead in blood and high lead levels are still coped by means of lead-removing food, health education, environmental intervention and special diet adjustment and balance, but there are no effective therapeutic medicines yet. At present, there are no medicines that can be used at an early stage to prevent chronic lead poisoning either. The search for lead poisoning antidotes and high lead levels therapeutic agents which have exact efficacy, high safety and low toxic and side effects, can be used in the early stage, have no peculiar smells, can be taken orally, and are not affected by kidney diseases, has clear application prospects.

In vivo pharmacokinetic study of lead showed that liver had a strong ability to secrete lead, and the concentration of lead in bile was 400 times to 1000 times that in blood. After being discharged into an intestinal tract through the bile, the lead was reabsorbed into the blood through enterohepatic circulation, so little lead was excreted. This suggests that a large amount of lead secreted by bile can be effectively from feces by taking a certain medicine orally to make the medicine combine with the lead in the digestive tract and block the enterohepatic circulation and reabsorption of the lead, so as to drive away the lead in food and body, thus achieving the purpose of removing the lead.

Anionic clay is a layered compound formed by the interaction of positively charged main laminates and interlayer anions through noncovalent bonds, and the structure of the anionic clay is schematically shown in FIG. 1. Cationic clays such as Montmorillonite and Bentonite are layered compounds formed by the interaction of negatively charged main laminates and interlayer cations through noncovalent bonds, which are completely different from the anionic clay. The anionic clay mostly exists in the form of layered composite hydroxides, including hydrotalcite and hydrotalcite-like compounds, almagate and almagate-like compounds, magaldrate and magaldrate-like compounds, almagcit, almagodrate, and the like, which are shown in Table I.

TABLE I
Category Examples of Anionic Clay

| Category | General formula | Example |
|---|---|---|
| Hydrotalcite and | M²⁺₍₁₋ₓ₎M³⁺ₓ(OH)₂ Aⁿ⁻_{x/n}•mH₂O, | Mg₆Al₂(OH)₁₆CO₃•4H₂O |
| hydrotalcite-like | wherein x = 0.200-0.333 | Mg₅ZnAl₂(OH)₁₆CO₃•4H₂O |
| compounds | | Mg₆Cr₂(OH)₁₆ SO₄•4H₂O |
| Almagate and | M²⁺_{(1-x/2)}M³⁺ₓ(OH)₂Aⁿ⁻_{2x/n}•mH₂O, | Mg₆Al₂(OH)₁₄(CO₃)₂•4H₂O |
| almagate-like | wherein x = 0.222-0.400 | Mg₆Al₂(OH)₁₄(SO₄)₂•4H₂O |
| compounds | | |
| Magaldrate and | M²⁺₍₁₋ₓ₎M³⁺ₓ(OH)₂(OH)_{x/5}Aⁿ⁻_{0.8x/n}•mH₂O, | Mg₁₀A₁₅(OH)₃₁(SO₄)₂•mH₂O |
| magaldrate-like | wherein x = 0.200-0.333 | Mg₁₀A₁₅(OH)₃₁(CO₃)₂•mH₂O |
| compounds | | |
| Others | | Mg₆Al₂(OH)₁₂(CO₃)₃•4H₂O |
| | | [Almagcit] |
| | | Mg₅Al₁₀(OH)₂₆O₅(SO₄)₂ |
| | | [Almagodrate] |

Note:
M²⁺ refers to divalent metal cation, M³⁺ refers to trivalent metal cation, An• refers to interlayer anion, n refers to valence value of anion, x refers to molar fraction of trivalent metal cation, and m refers to quantity of interlayer water.

Anionic clay has structural characteristics such as adjustability of metal ion composition of the main laminates, adjustability of charge density and distribution of the main laminates, adjustability of interlayer anion categories and quantities, adjustability of interlayer space, adjustability of interaction between the main laminates and the interlayer anions, and the like, which make the anionic clay have properties such as acid-base, adsorption, ion exchange, thermal stability, flame retardancy, ultraviolet barrier, or the like, and may be widely applied to catalysis, medicine, ion exchange and adsorption, flame retardancy, ultraviolet absorption and other fields. Almagate [Al₂Mg₆(OH)₁₄(CO₃)₂.4H₂O], magaldrate [Al₅Mg₁₀(OH)₃₁(SO₄)₂.mH₂O] and hydrotalcite [Al₂Mg₆(OH)₁₆CO₃.4H₂O] are used as antacids, which have the characteristics of high safety, little toxic and side effects and no peculiar smell, may be taken orally, and are widely used in the field of medicine.

In view of the special layered structure of the anionic clay, the anionic clay is often used to adsorb and remove anions and organic matters, and may be used as an excellent cation adsorption material at the same time. Up to now, however, there is no prior art that discloses a use of the anionic clay in preparing a lead-removing medicine.

### SUMMARY

In view of the problems in the prior art, the object of the present invention is to provide a use of anionic clay in preparing a lead-removing medicine.

The object of the present invention may be achieved by the following technical solutions.

The present invention provides a use of anionic clay in preparing a lead-removing medicine.

The anionic clay includes such compounds as hydrotalcite and hydrotalcite-like compounds, almagate and almagate-like compounds, magaldrate and magaldrate-like compounds, almagcit, almagodrate, and the like.

As one aspect of the present invention, a composition or compound medicine is formed by taking anionic clay as an effective ingredient and matched with a pharmaceutically acceptable therapeutic agent, which may be used for preparing a lead-removing medicine.

As another aspect of the present invention, a pharmaceutically acceptable adjuvant is added into the anionic clay or the composition or compound medicine thereof to prepare a capsule, a tablet, a powder, a granule, a suspension or a suspensoid, which may realize a use of preparing a lead-removing medicine.

Compared with the prior art, the present invention has the beneficial effects as follows.

1) The medicinal use of the anionic clay according to the present invention can effectively prevent and treat lead poisoning and high lead levels.

2) The medicinal use of the anionic clay according to the present invention may be used as the therapeutic medicine for the lead poisoning of Level III and below in the international diagnostic standards for lead in blood and high lead levels, breaking through the treatment limitations of the existing lead-removing agent, greatly advancing the lead-removing treatment time, significantly expanding the lead-removing treatment range, changing the current situation that there is no effective therapeutic medicine for the above diseases, and having positive clinical significance.

3) The almagate, the magaldrate and the hydrotalcite in the anionic clay have been used as antacids in clinic and proved to be safe. On the basis of the present invention, the anionic clay may be expected to be used for clinical prevention and treatment of lead poisoning and high lead levels.

According to the present invention, the indication that anionic clay is used for removing lead in organisms is first discovered.

### BRIEF DESCRIPTION OF THE DRAWINGS

FIG. 1 is a schematic structural diagram of anionic clay.

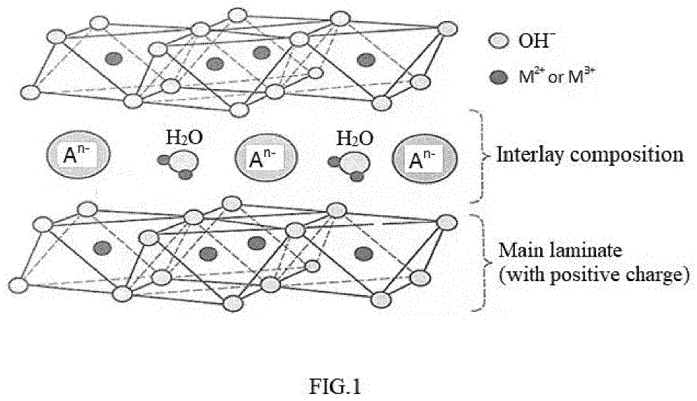

### DETAILED DESCRIPTION

The present invention will be further described hereinafter in detail with reference to the embodiments. The following embodiments will help those skilled in the art to further understand the present invention, but will not limit the present invention in any way. It should be pointed out that for those of ordinary skills in the art, several adjustments and improvements can be made without departing from the concept of the present invention. These adjustments and improvements all belong to the protection scope of the present invention.

### Embodiment 1 In Vitro Study on Adsorption of Lead by Hydrotalcite

### 1.1 Medicine and Reagent

Hydrotalcite (Kyowa Chemical Industry Co., Ltd., Japan); Lead acetate (Sinopharm); potassium dihydrogen phosphate (Sinopharm); and sodium hydroxide (Sinopharm).

### 1.2 Reagent Preparation

Buffer solution: 6.8 g of potassium dihydrogen phosphate was weighed and dissolved with 500 mL of water, and the pH was adjusted to 6.8 with 0.4% sodium hydroxide solution; then water was added to a constant volume of 1,000 mL.

Lead nitrate solution: 0.1599 g of lead nitrate was weighed and placed in a 1000 mL volumetric flask, added with 50 mL of buffer solution to dissolve, and diluted to scale with the buffer solution. Every milliliter contained 100 μg of Pb.

### 1.3 In Vitro Study on Adsorption of Lead by Hydrotalcite

10 mL of lead nitrate solution (100 μg/mL) was weighed, placed in a 100 mL volumetric flask, added with buffer solution and diluted to scale, and then shaken evenly (each milliliter contained 10 μg of lead). Then, the mixture was transferred to a container, and stirred at a constant temperature of 37° C. under airtight conditions for 30 minutes.

10 mg of hydrotalcite was weighed and added into the above-mentioned lead solution (10 μg/mL) to form a mixed system, and stirred at 37° C. under airtight conditions for 2 hours. The pH values of the mixed system were determined at the 15^{th} minute, the 30^{th} minute, the 45^{th} minute, the 60^{th} minute and the 120^{th} minute respectively, and then samples were taken and filtered to determine the concentration of lead ions in the filtrate. As a reference, the lead solution stock solution (10 μg/mL) without hydrotalcite was filtered and the lead ion concentration therein was detected. Lead was detected by plasma atomic emission spectrometry. The detection results are shown in Table 1.

TABLE 1
In vitro study result on adsorption of lead by hydrotalcite

| | | | | | | |
|---|---|---|---|---|---|---|
| Time (min) | — | 15 | 30 | 45 | 60 | 120 |
| Lead concentration | 9.96 | 0.56 | 0.51 | 0.34 | 0.14 | 0.09 |
| (μg/mL) | | | | | | |
| Adsorption rate | — | 94.4 | 94.9 | 96.6 | 98.6 | 99.1 |
| (%) | | | | | | |

It can be seen from Table 1 that the adsorption rate of hydrotalcite for the lead is over 90% in the 15th minute, which indicates that the hydrotalcite has a strong adsorption capacity for the lead in the solution.

### Embodiment 2 Study on the Prevention and Treatment of Chronic Lead Poisoning in Rats with Hydrotalcite

### 2.1 Medicine and Reagent

Hydrotalcite (Kyowa Chemical Industry Co., Ltd., Japan); Lead acetate (Sinopharm); perchloric acid (Sinopharm); and sodium dimercaptosuccinate (Shanghai New Asiatic Pharmaceutical).

### 2.2 Experimental Animal

Male SD rats, weighing 130 g to 150 g, provided by the Animal Center of Shanghai Xipuer-Bikai Laboratory Animal Co., Ltd. (Certificate No.: SCXK 2008-0016).

### 2.3 Main Instrument

Assay balance (METTLER TOLEDO, Swiss, and model: PL 601-L); rat scale (Nanjing Emmanuel Instrument Equipment Co., Ltd.); and series 7700 ICP-MS (Agilent, USA).

### 2.4 Experimental Method

(1) Chronic Rat Lead Poisoning Model and Administration for Prevention

32 male SD rats were randomly divided into four groups including a normal group, a model group, a high-dose hydrotalcite group and a low-dose hydrotalcite group according to their body weights, and each group included 8 rats. Except the rats in the normal group were given ultrapure water, the rats in the model group and the hydrotalcite groups of each dose drank 0.5% lead acetate ultrapure water solution for 30 days, resulting in chronic lead-exposed models. From the 4^{th} day of modeling, the rats in the high-dose hydrotalcite group and the low-dose hydrotalcite group were given a high dose of 0.6 g/kg and a low dose of 0.3 g/kg, respectively (the hydrotalcite and deionized water were mixed to make suspension, which was given by intragastric administration with equal volume). The rats in the normal group were given equal volume of 0.5% CMC-Na solution by intragastric administration. All the subjects subjected to intragastric administration were given in equal volume twice a day for 30 days. After 30 days, blood samples were collected from aorta abdominalis, and the rats were sacrificed under excessive anesthesia. Brain, liver and femur samples were collected and stored in liquid nitrogen for later use. After wet digestion, the blood samples and tissues of each group were tested for content of lead by inductively coupled plasma mass spectrometry (ICP-MS), and the lead-removing rates were counted. The results were shown in Tables 2 to 5.

(2) Chronic Rat Lead Poisoning Model and Administration for Treatment

40 male SD rats were randomly divided into five groups including a normal group, a model group, a positive medicine group (sodium dimercaptosuccinate group), a high-dose hydrotalcite group and a low-dose hydrotalcite group according to their body weights, and each group included 8 rats. Except the rats in the normal group were given ultrapure water, the rats in the model group and the hydrotalcite groups of each dose drank 0.5% lead acetate ultrapure water solution for 30 days, resulting in chronic lead-exposed models. After the model was successfully established, the lead was stopped and the rates were fed with distilled water from the 31^{st} day. The rats in the normal group were given an equal volume of 0.5% CMC-Na solution by intragastric administration, while the rats in the high-dose hydrotalcite group and the low-dose hydrotalcite group were given a high dose of 0.6 g/kg and a low dose of 0.3 g/kg respectively by intragastric administration. The rats in the positive medicine group were given a dose of 10 mg/kg of sodium dimercaptosuccinate by intragastric administration, and all the intragastric subjects were given in equal volume twice a day for 30 days. On the 60^{th} day, blood samples were collected. Brain, liver and femur samples were collected and stored in liquid nitrogen for later use. After wet digestion, the blood samples and tissues of each group were tested for content of lead by inductively coupled plasma mass spectrometry (ICP-MS), and the lead-removing rates were counted. The results were shown in Tables 6 to 9.

All the data were processed by SPSS 20.0 statistical software. Every group of measurement data was expressed by mean±relative standard deviation (mean±SD), and all of which were tested for homogeneity of variance by Levene method first. If the variances were homogeneous, single factor analysis of variance was used among multiple groups, and two-sample T test was used between two groups. If the hypothesis of parameter test was unreasonable, Kruskal-Wallis test was used among multiple groups, while Wilcoxon test was used between two groups. p<0.05 or p<0.01 was statistically significant.

### 2.5 Experimental Result

### 2.5.1 Effects of Administration of Hydrotalcite for Prevention on Lead in Blood, Lead in Brain and Lead in Bones in Chronic Rat Lead Poisoning Model

The effects of the administration of hydrotalcite for prevention on the lead in blood, the lead in brain and the lead in bones in the chronic rat lead poisoning model are shown in Tables 2 to 4.

TABLE 2
Effects of hydrotalcite on content of lead in blood in rats with chronic
lead poisoning (mean ± SD)

| | | | Content of |
|---|---|---|---|
| Group | Number of animals (piece) | Dose (g/kg) | lead (μg/g) |
| Normal group | 8 | — | 0.033 ± 0.01 |
| Model group | 8 | — | 0.064 ± 0.05^{##} |
| Low-dose | 8 | 0.3 | 0.045 ± 0.03 |
| hydrotalcite group | | | |
| High-dose | 8 | 0.6 | 0.024 ± 0.01* |
| hydrotalcite group | | | |

Note:
compared with the normal group: ^{##}p < 0.01; and
compared with the model group: *p < 0.05.

TABLE 3
Effects of hydrotalcite on content of lead in brain in rats with chronic
lead poisoning (mean ± SD)

| | | | Content of |
|---|---|---|---|
| Group | Number of animals (piece) | Dose (g/kg) | lead (μg/g) |
| Normal | 8 | — | 0.047 ± 0.06 |
| Model group | 8 | — | 0.694 ± 0.10^{##} |
| Low-dose | 8 | 0.3 | 0.593 ± 0.22 |
| hydrotalcite group | | | |
| High-dose | 8 | 0.6 | 0.540 ± 0.14* |
| hydrotalcite group | | | |

Note:
compared with the normal group: ^{##}p < 0.01; and
compared with the model group: *p < 0.05.

TABLE 4
Effects of hydrotalcite on femur content of lead in
rats with chronic lead poisoning (mean ± SD)

| | Number | | Content |
|---|---|---|---|
| | of animals | Dose | of lead |
| Group | (piece) | (g/kg) | (μg/g) |
| Normal group | 8 | — | 0.231 ± 0.16 |
| Model group | 8 | — | 55.062 ± 7.49^{##} |
| Low-dose | 8 | 0.3 | 57.740 ± 14.05 |
| hydrotalcite | | | |
| group | | | |
| High-dose | 8 | 0.6 | 46.252 ± 6.46* |
| hydrotalcite | | | |
| group | | | |

Note:
compared with the normal group: ^{##}p <0.01;
and compared with the model group: *p <0.05.

The results are shown in Tables 2, 3 and 4. Compared with the normal group, the contents of the lead in blood, the lead in brain and the lead in bones in the model group and the hydrotalcite groups are significantly increased, indicating that modeling with this method is successful. By comparing the low-dose hydrotalcite group, the high-dose hydrotalcite group and the model group, the results show that the contents of the lead in blood, the lead in brain and the lead in bones of the high-dose hydrotalcite group are significantly lower than that of the model group, and are dose-dependent. Furthermore, the lead-removing rates of the hydrotalcite on various tissues of rats with chronic lead poisoning were calculated. The results are shown in Table 5. The effective removing rates of the hydrotalcite on the lead in brain at low and high doses are 26.63% and 28.29% respectively. The effective removing rates of the lead in blood at low and high doses are 42.84% and 59.04% respectively. The effective removing rates of the lead in bones at low and high doses are 0 and 7.52% respectively.

TABLE 5
Effective removing rates of hydrotalcite on lead in
various tissues of rats with chronic lead poisoning

| | Removing rate (%) | | |
|---|---|---|---|
| | Lead in | Lead in | Lead in |
| Group | brain | blood | bones |
| Low-dose group (0.3 g/kg) | 26.63 | 42.84 | 0 |
| High-dose group (0.6 g/kg) | 28.29 | 59.04 | 7.52 |

### 2.5.2 Effects of Administration of Hydrotalcite for Treatment on Lead in Blood, Lead in Brain and Lead in Bones in Chronic Rat Lead Poisoning Model

The effects of the administration of hydrotalcite for prevention on the lead in blood, the lead in brain and the lead in bones in the chronic rat lead poisoning model are shown in Tables 6 to 8.

TABLE 6
Effects of hydrotalcite on content of lead in blood in rats
with chronic lead poisoning (mean ± SD) on the 60^{th} day

| | Number | | Content |
|---|---|---|---|
| | of animals | Dose | of lead |
| Group | (piece) | (g/kg) | (μg/g) |
| Normal group | 8 | — | 0.014 ± 0.03 |
| Model group | 8 | — | 0.075 ± 0.02^{##} |
| Low-dose | 8 | 0.3 | 0.086 ± 0.01 |
| hydrotalcite | | | |
| group | | | |
| High-dose | 8 | 0.6 | 0.055 ± 0.01* |
| hydrotalcite | | | |
| group | | | |
| Sodium | 8 | 0.01 | 0.058 ± 0.01 |
| dimercaptosuccinate | | | |
| group | | | |

Note:
compared with the normal group: ^{##}p <0.01;
and compared with the model group: *p <0.05.

TABLE 7
Effects of hydrotalcite on content of lead in brain in rats
with chronic lead poisoning (mean ± SD) on the 60^{th} day

| | Number | | Content |
|---|---|---|---|
| | of animals | Dose | of lead |
| Group | (piece) | (g/kg) | (μg/g) |
| Normal group | 8 | — | 0.026 ± 0.02 |
| Model group | 8 | — | 0.446 ± 0.40^{##} |
| Low-dose | 8 | 0.3 | 0.228 ± 0.08 |
| hydrotalcite | | | |
| group | | | |
| High-dose | | 0.6 | 0.079 ± 0.01* |
| hydrotalcite | | | |
| group | | | |
| Sodium | 8 | 0.01 | 0.129 ± 0.03 |
| dimercaptosuccinate | | | |
| group | | | |

Note:
compared with the normal group: ^{##}p <0.05;
and compared with the model group: *p <0.05.

TABLE 8
Effects of hydrotalcite on femur content of lead in rats
with chronic lead poisoning (mean ± SD) on the 60^{th} day

| | Number | | Content |
|---|---|---|---|
| | of animals | Dose | of lead |
| Group | (piece) | (g/kg) | (μg/g) |
| Normal group | 8 | — | 0.444 ± 0.47 |
| Model group | 8 | — | 25.036 ± 5.13^{##} |
| Low-dose | 8 | 0.3 | 25.379 ± 4.22 |
| hydrotalcite | | | |
| group | | | |
| High-dose | 8 | 0.6 | 17.430 ± 4.21* |
| hydrotalcite | | | |
| group | | | |
| Sodium | 8 | 0.01 | 32.085 ± 4.73 |
| dimercaptosuccinate | | | |
| group | | | |

Note:
compared with the normal group: ^{##}p <0.01;
and compared with the model group: *p <0.05.

The results are shown in Tables 6, 7 and 8. Compared with the normal group, the contents of the lead in blood, the lead in brain and the lead in bones in the model group, the hydrotalcite groups and the positive medicine group (sodium dimercaptosuccinate group) are significantly increased, indicating that modeling with this method is successful. By comparing the model group, the low-dose hydrotalcite group and the high-dose hydrotalcite group, the results show that the contents of the lead in blood, the lead in brain and the lead in bones of the high-dose hydrotalcite group are significantly lower than that of the model group, and are dose-dependent. Furthermore, the lead-removing rates among the administration groups were analyzed. The details are shown in Table 9. The effective removing rate of the hydrotalcite administration group for treatment on the lead in brain is 14.91% at high dose, the effective removing rates of the hydrotalcite administration group for treatment on the lead in brain at low and high doses are 26.15% and 54.85% respectively, and the effective removing rate of the hydrotalcite administration group for treatment on the lead in bones at high dose is 19.10%.

TABLE 9
Effective removing rates of hydrotalcite on lead in
various tissues of rats with chronic lead poisoning

| | Removing rate (%) | | |
|---|---|---|---|
| | Positive | Low-dose | High-dose |
| | medicine | hydrotalcite | hydrotalcite |
| Item | group | group | group |
| Lead in | 6.77 | 0.00 | 14.91 |
| blood | | | |
| Lead in | 26.74 | 26.15 | 54.85 |
| brain | | | |
| Lead in | 0.00 | 0.00 | 19.10 |
| bones | | | |

### 2.6 Experimental Conclusion

According to the result judgment standard of “Method for the Assessment of Alleviating Lead Excretion Function” promulgated by China, compared with the model group, the lead in blood, the lead in brain and the lead in bones in the hydrotalcite groups are reduced significantly, and thus, it can be judged that the animal experimental results of the tested sample are positive. The efficacy results of the present invention show that the administration of hydrotalcite for prevention and treatment can effectively improve the contents of the lead in blood, the lead in brain and the lead in bones in the rats with chronic lead poisoning.

In conclusion, the hydrotalcite has the pharmacological action of removing lead, and has good preventive and therapeutic effects when being used as a lead-removing agent.

### Embodiment 3 In Vitro Study on Adsorption of Lead by Magaldrate

### 3.1 Medicine and Reagent

Magaldrate (Zhejiang CR Sanjiu Pharmaceutical Group Limited); Lead acetate (Sinopharm); potassium dihydrogen phosphate (Sinopharm); and sodium hydroxide (Sinopharm).

### 3.2 Reagent Preparation

Buffer solution: 6.8 g of potassium dihydrogen phosphate was weighed and added with 500 mL of water. The pH was adjusted to 6.8 with 0.4% sodium hydroxide solution; then water was added to a constant volume of 1,000 mL.

Lead solution: 0.1599 g of lead nitrate was weighed and placed in a 1000 mL volumetric flask, added with 50 mL of buffer solution to dissolve, and diluted to scale with the buffer solution. Every milliliter contained 100 μg of Pb.

### 3.3 In Vitro Study on Adsorption of Lead by Magaldrate

10 mL of lead solution (100 μg/mL) was weighed, placed in a 100 mL volumetric flask, added with buffer solution and diluted to scale, and then shaken evenly (each milliliter contained 10 μg of Pb). Then, the mixture was transferred to a container, and stirred at a constant temperature of 37° C. under airtight conditions for 30 minutes.

10 mg of magaldrate sample was weighed and added into the above-mentioned lead solution (10 μg/mL) to form a mixed system, and stirred at 37° C. under airtight conditions for 2 hours. The samples were taken and filtered at the 15^{th} minute, the 30^{th} minute, the 45^{th} minute, the 60^{th} minute and 120^{th} minute respectively. The pH value of the adsorbed solution was determined and the concentration of the lead irons was determined. As a reference, the lead solution stock solution (10 μg/mL) without magaldrate was filtered and the lead ion concentration therein was detected. Lead was detected by plasma atomic emission spectrometry. The detection results are shown in Table 10.

TABLE 10
In vitro study result on adsorption of lead by magaldrate

| | | | | | | |
|---|---|---|---|---|---|---|
| Time (min) | — | 15 | 30 | 45 | 60 | 120 |
| Lead concentration | 10.03 | 1.75 | 1.49 | 1.32 | 1.18 | 0.92 |
| (μg/mL) | | | | | | |
| Adsorption rate | — | 82.5 | 85.1 | 86.8 | 88.2 | 90.8 |
| (%) | | | | | | |

It can be seen from Table 10 that the adsorption rate of the magaldrate on the lead is higher than 80% at the 15^{th} minute, and the adsorption effect is obvious.

### Embodiment 4 Study on the Prevention and Treatment of Chronic Lead Poisoning in Rats with Magaldrate

### 4.1 Medicine and Reagent

Magaldrate (Zhejiang CR Sanjiu Pharmaceutical Group Limited); Lead acetate (Sinopharm); perchloric acid (Sinopharm); and sodium dimercaptosuccinate (Shanghai New Asiatic Pharmaceutical).

### 4.2 Experimental Animal

Male SD rats, weighing 130 g to 150 g, provided by the Animal Center of Shanghai Xipuer-Bikai Laboratory Animal Co., Ltd. (Certificate No.: SCXK 2008-0016).

### 4.3 Main Instrument

Assay balance (METTLER TOLEDO, Swiss, and model: PL 601-L); rat scale (Nanjing Emmanuel Instrument Equipment Co., Ltd.); and series 7700 ICP-MS (Agilent, USA).

### 4.4 Experimental Method

(1) Chronic Rat Lead Poisoning Model and Administration for Prevention

32 male SD rats were randomly divided into four groups including a normal group, a model group, a high-dose magaldrate group and a low-dose magaldrate group according to their body weights, and each group included 8 rats. Except the rats in the normal group were given ultrapure water, the rats in the model group and the magaldrate groups of each dose drank 0.5% lead acetate ultrapure water solution for 30 days, resulting in chronic lead-exposed models. From the 4^{th} day of modeling, the rats in the high-dose magaldrate group and the low-dose magaldrate group were given a high dose of 0.6 g/kg and a low dose of 0.3 g/kg, respectively (the magaldrate and deionized water were mixed to make suspension, which was given by intragastric administration with equal volume). The rats in the normal group were given equal volume of 0.5% CMC-Na solution by intragastric administration. All the subjects subjected to intragastric administration were given in equal volume twice a day for 30 days. After 30 days, blood samples were collected from aorta abdominalis, and the rats were sacrificed under excessive anesthesia. Brain, liver and femur samples were collected and stored in liquid nitrogen for later use. After wet digestion, the blood samples and tissues of each group were tested for content of lead by inductively coupled plasma mass spectrometry (ICP-MS), and the lead-removing rates were counted. The results were shown in Tables 11 to 14.

(2) Chronic Rat Lead Poisoning Model and Administration for Treatment

40 male SD rats were randomly divided into five groups including a normal group, a model group, a positive medicine group (sodium dimercaptosuccinate group), a high-dose magaldrate group and a low-dose magaldrate group according to their body weights, and each group included 8 rats. Except the rats in the normal group were given ultrapure water, the rats in the model group, the positive medicine group, and the magaldrate groups of each dose drank 0.5% lead acetate ultrapure water solution for 30 days, resulting in chronic lead-exposed models. After the model was successfully established, the lead was stopped and the rates were fed with distilled water from the 31^{st} day. The rats in the normal group were given an equal volume of 0.5% CMC-Na solution by intragastric administration, while the rats in the high-dose magaldrate group and the low-dose magaldrate group were given a high dose of 0.6 g/kg and a low dose of 0.3 g/kg respectively by intragastric administration. The rats in the positive medicine group were given a dose of 10 mg/kg of sodium dimercaptosuccinate by intragastric administration, and all the intragastric subjects were given in equal volume twice a day for 30 days. On the 60th day, blood samples were collected. Brain, liver and femur samples were collected and stored in liquid nitrogen for later use. After wet digestion, the blood samples and tissues of each group were tested for content of lead by inductively coupled plasma mass spectrometry (ICP-MS), and the lead-removing rates were counted. The results were shown in Tables 15 to 18.

All the data were processed by SPSS20.0 statistical software. Every group of measurement data was expressed by mean±relative standard deviation (mean±SD), and subjected to T test. p<0.05 was statistically significant.

### 4.5 Experimental Result

4.5.1 Effects of Administration of Magaldrate for Prevention on Lead in Blood, Lead in Brain and Lead in Bones in Chronic Rat Lead Poisoning Model

The effects of the administration of magaldrate for prevention on the lead in blood, the lead in brain and the lead in bones in the chronic rat lead poisoning model are shown in Tables 11 to 13.

TABLE 11
Effects of magaldrate on content of lead in blood in rats
with chronic lead poisoning (mean ± SD)

| | Number | | Content |
|---|---|---|---|
| | of animals | Dose | of lead |
| Group | (piece) | (g/kg) | (μg/g) |
| Normal group | 8 | — | 0.023 ± 0.02 |
| Model group | 8 | — | 0.072 ± 0.07^{##} |
| Low-dose | 8 | 0.3 | 0.055 ± 0.05 |
| magaldrate | | | |
| group | | | |
| High-dose | 8 | 0.6 | 0.042 ± 0.03* |
| magaldrate | | | |
| group | | | |

Note:
compared with the normal group: ^{##}p <0.01;
and compared with the model group: *p <0.05.

TABLE 12
Effects of magaldrate on content of lead in brain in rats
with chronic lead poisoning (mean ± SD)

| | Number | | Content |
|---|---|---|---|
| | of animals | Dose | of lead |
| Group | (piece) | (g/kg) | (μg/g) |
| Normal group | 8 | — | 0.032 ± 0.03 |
| Model group | 8 | — | 0.576 ± 0.08^{##} |
| Low-dose | 8 | 0.3 | 0.521 ± 0.15 |
| magaldrate | | | |
| group | | | |
| High-dose | 8 | 0.6 | 0.487 ± 0.12* |
| magaldrate | | | |
| group | | | |

Note:
compared with the normal group: ^{##}p <0.01;
and compared with the model group: *p <0.05.

TABLE 13
Effects of magaldrate on femur content of lead in rats
with chronic lead poisoning (mean ± SD)

| | Number | | Content |
|---|---|---|---|
| | of animals | Dose | of lead |
| Group | (piece) | (g/kg) | (μg/g) |
| Normal group | 8 | — | 0.362 ± 0.27 |
| Model group | 8 | — | 48.792 ± 5.47^{##} |
| Low-dose | 8 | 0.3 | 50.010 ± 6.18 |
| magaldrate | | | |
| group | | | |
| High-dose | 8 | 0.6 | 45.316 ± 5.22* |
| magaldrate | | | |
| group | | | |

Note:
compared with the normal group: ^{##}p <0.01;
and compared with the model group: *p <0.05.

The results are shown in Tables 11, 12 and 13. Compared with the normal group, the contents of the lead in blood, the lead in brain and the lead in bones in the model group and the magaldrate groups are significantly increased, indicating that modeling with this method is successful. By comparing the low-dose magaldrate group, the high-dose magaldrate group and the model group, the results show that the contents of the lead in blood, the lead in brain and the lead in bones of the high-dose magaldrate group are significantly lower than that of the model group, and are dose-dependent. Furthermore, the lead-removing rates of the magaldrate on various tissues of rats with chronic lead poisoning were calculated. The results are shown in Table 14. The effective rates of the magaldrate on the lead in brain at low and high doses are 9.55% and 15.45% respectively. The effective removing rates of the lead in blood at low and high doses are 23.61% and 41.67% respectively. The effective removing rates of the lead in bones at low and high doses are 0 and 7.12% respectively.

TABLE 14
Effective removing rates of magaldrate on lead
in various tissues of rats with chronic lead poisoning

| | Removing rate (%) | | |
|---|---|---|---|
| | Lead in | Lead in | Lead in |
| Group | brain | blood | bones |
| Low-dose group (0.3 g/kg) | 9.55 | 23.61 | 0 |
| High-dose group (0.6 g/kg) | 15.45 | 41.67 | 7.12 |

4.5.2 The effects of the administration of magaldrate for treatment on the lead in blood, the lead in brain and the lead in bones in the chronic rat lead poisoning model are shown in Tables 15 to 17.

TABLE 15
Effects of magaldrate on content of lead in blood in rats
with chronic lead poisoning (mean ± SD) on the 60^{th} day

| | Number | | Content |
|---|---|---|---|
| | of animals | Dose | of lead |
| Group | (piece) | (g/kg) | (μg/g) |
| Normal group | 8 | — | 0.010 ± 0.04 |
| Model group | 8 | — | 0.080 ± 0.03^{##} |
| Low-dose | 8 | 0.3 | 0.096 ± 0.02 |
| magaldrate | | | |
| group | | | |
| High-dose | 8 | 0.6 | 0.066 ± 0.01* |
| magaldrate | | | |
| group | | | |
| Sodium | 8 | 0.01 | 0.069 ± 0.01 |
| dimercaptosuccinate | | | |
| group | | | |

Note:
compared with the normal group: ^{##}p <0.01;
and compared with the model group: *p <0.05.

TABLE 16
Effects of magaldrate on content of lead in brain in rats
with chronic lead poisoning (mean ± SD) on the 60^{th} day

| | Number | | Content |
|---|---|---|---|
| | of animals | Dose | of lead |
| Group | (piece) | (g/kg) | (μg/g) |
| Normal group | 8 | — | 0.028 ± 0.03 |
| Model group | 8 | — | 0.516 ± 0.48^{##} |
| Low-dose | 8 | 0.3 | 0.427 ± 0.13 |
| magaldrate | | | |
| group | | | |
| High-dose | 8 | 0.6 | 0.239 ± 0.02* |
| magaldrate | | | |
| group | | | |
| Sodium | 8 | 0.01 | 0.218 ± 0.04 |
| dimercaptosuccinate | | | |
| group | | | |

Note:
compared with the normal group: ^{##}p <0.05;
and compared with the model group: *p <0.05.

TABLE 17
Effects of magaldrate on femur content of lead in rats
with chronic lead poisoning (mean ± SD) on the 60^{th} day

| | Number | | Content |
|---|---|---|---|
| | of animals | Dose | of lead |
| Group | (piece) | (g/kg) | (μg/g) |
| Normal group | 8 | — | 0.398 ± 0.366 |
| Model group | 8 | — | 26.864 ± 4.12^{##} |
| Low-dose | 8 | 0.3 | 27.124 ± 3.97 |
| magaldrate | | | |
| group | | | |
| High-dose | 8 | 0.6 | 23.378 ± 3.26* |
| magaldrate | | | |
| group | | | |
| Sodium | 8 | 0.01 | 35.006 ± 3.92 |
| dimercaptosuccinate | | | |
| group | | | |

Note:
compared with the normal group: ^{##}p <0.01;
and compared with the model group: *p <0.05.

The results are shown in Tables 15, 16 and 17. Compared with the normal group, the contents of the lead in blood, the lead in brain and the lead in bones in the model group, the magaldrate groups and the positive medicine group (sodium dimercaptosuccinate group) are significantly increased, indicating that modeling with this method is successful. By comparing the model group, the low-dose magaldrate group and the high-dose magaldrate group, the results show that the contents of the lead in blood, the lead in brain and the lead in bones of the high-dose magaldrate group are significantly lower than that of the model group, and are dose-dependent. Furthermore, the lead-removing rates among the administration groups were analyzed. The details are shown in Table 18. The effective removing rate of the magaldrate administration group for treatment on the lead in brain is 17.50% at high dose, the effective removing rates of the magaldrate administration group for treatment on the lead in brain at low and high doses are 17.25% and 53.68% respectively, and the effective removing rate of the magaldrate administration group for treatment on the lead in bones at high dose is 12.98%.

TABLE 18
Effective removing rates of magaldrate on lead in
various tissues of rats with chronic lead poisoning

| | Removing rate (%) | | |
|---|---|---|---|
| | Positive | low-dose | High-dose |
| | medicine | magaldrate | magaldrate |
| Item | group | group | group |
| Lead in | 13.75 | 0.00 | 17.50 |
| blood | | | |
| Lead in | 57.75 | 17.25 | 53.68 |
| brain | | | |
| Lead in | 0.00 | 0.00 | 12.98 |
| bones | | | |

### 4.6 Experimental Conclusion

According to the result judgment standard of “Method for the Assessment of Alleviating Lead Excretion Function” promulgated by China, compared with the model group, the lead in blood, the lead in brain and the lead in bones in the magaldrate groups are reduced significantly, and thus, it can be judged that the animal experimental results of the tested sample are positive. The efficacy results of the present invention show that the administration of magaldrate for prevention and treatment can effectively improve the contents of the lead in blood, the lead in brain and the lead in bones in the rats with chronic lead poisoning.

In conclusion, the magaldrate has the pharmacological action of removing lead, and has good preventive and therapeutic effects when being used as a lead-removing agent.

### Embodiment 5 In Vitro Study on Adsorption of Lead by Almagate

### 5.1 Medicine and Reagent

Almagate (manufactured by IL-YANG PHARM. CO., LTD, KOREA); lead acetate (Sinopharm); potassium dihydrogen phosphate (Sinopharm); and sodium hydroxide (Sinopharm).

### 5.2 Reagent Preparation

Buffer solution: 6.8 g of potassium dihydrogen phosphate was weighed and added with 500 mL of water. The pH was adjusted to 6.8 with 0.4% sodium hydroxide solution; and then water was added to a constant volume of 1,000 mL.

Lead solution: 0.1599 g of lead nitrate was weighed and placed in a 1000 mL volumetric flask, added 50 mL of buffer solution to dissolve, and diluted to scale with the buffer solution. Every milliliter contained 100 μg of Pb.

### 5.3 In Vitro Study on Adsorption of Lead by Almagate

10 mL of lead solution (100 μg/mL) was weighed, placed in a 100 mL volumetric flask, added with buffer solution and diluted to scale, and then shaken evenly (each milliliter contained 10 μg of Pb). Then, the mixture was transferred to a container, and stirred at a constant temperature of 37° C. under airtight conditions for 30 minutes.

10 mg of almagate sample was weighed and added into the above-mentioned lead solution (10 μg/mL) to form a mixed system, and stirred at 37° C. under airtight conditions for 2 hours. The samples were taken and filtered at the 15^{th} minute, the 30^{th} minute, the 45^{th} minute, the 60^{th} minute and 120^{th} minute respectively. The pH value of the adsorbed solution was determined and the concentration of the lead irons was determined. As a reference, the lead solution stock solution (10 μg/mL) without almagate was filtered and the lead ion concentration therein was detected. Lead was detected by plasma atomic emission spectrometry. The detection results are shown in Table 19.

TABLE 19
In vitro study result on adsorption of lead by almagate

| | | | | | | |
|---|---|---|---|---|---|---|
| Time (min) | — | 15 | 30 | 45 | 60 | 120 |
| Lead concentration | 9.91 | 1.08 | 0.76 | 0.54 | 0.37 | 0.28 |
| (μg/mL) | | | | | | |
| Adsorption rate | — | 89.1 | 92.3 | 94.5 | 96.3 | 97.2 |
| (%) | | | | | | |

It can be seen from Table 19 that the adsorption rate of the almagate on the lead is higher than 80% at the 15th minute, and the adsorption effect is obvious.

### Embodiment 6 Study on the Prevention and Treatment of Chronic Lead Poisoning in Rats with Almagate

### 6.1 Medicine and Reagent

Almagate (manufactured by IL-YANG PHARM. CO., LTD, KOREA); lead acetate (Sinopharm); perchloric acid (Sinopharm); and sodium dimercaptosuccinate (Shanghai New Asiatic Pharmaceutical).

### 6.2 Experimental Animal

Male SD rats, weighing 130 g to 150 g, provided by the Animal Center of Shanghai Xipuer-Bikai Laboratory Animal Co., Ltd. (Certificate No.: SCXK 2008-0016).

### 6.3 Main Instrument

Assay balance (METTLER TOLEDO, Swiss, and model: PL 601-L); rat scale (Nanjing Emmanuel Instrument Equipment Co., Ltd.); and series 7700 ICP-MS (Agilent, USA).

### 6.4 Experimental Method

(1) Chronic Rat Lead Poisoning Model and Administration for Prevention

32 male SD rats were randomly divided into four groups including a normal group, a model group, a high-dose almagate group and a low-dose almagate group according to their body weights, and each group included 8 rats. Except the rats in the normal group were given ultrapure water, the rats in the model group and the almagate groups of each dose drank 0.5% lead acetate ultrapure water solution for 30 days, resulting in chronic lead-exposed models. From the 4th day of modeling, the rats in the high-dose almagate group and the low-dose almagate group were given a high dose of 0.6 g/kg and a low dose of 0.3 g/kg, respectively (the almagate and deionized water were mixed to make suspension, which was given by intragastric administration with equal volume). The rats in the normal group were given equal volume of 0.5% CMC-Na solution by intragastric administration. All the subjects subjected to intragastric administration were given in equal volume twice a day for 30 days. After 30 days, blood samples were collected from aorta abdominalis, and the rats were sacrificed under excessive anesthesia. Brain, liver and femur samples were collected and stored in liquid nitrogen for later use. After wet digestion, the blood samples and tissues of each group were tested for content of lead by inductively coupled plasma mass spectrometry (ICP-MS), and the lead-removing rates were counted. The results were shown in Tables 20 to 23.

(2) Chronic Rat Lead Poisoning Model and Administration for Treatment

40 male SD rats were randomly divided into five groups including a normal group, a model group, a positive medicine group (sodium dimercaptosuccinate group), a high-dose almagate group and a low-dose almagate group according to their body weights, and each group included 8 rats. Except the rats in the normal group were given ultrapure water, the rats in the model group, the positive medicine group, and the almagate groups of each dose drank 0.5% lead acetate ultrapure water solution for 30 days, resulting in chronic lead-exposed models. After the model was successfully established, the lead was stopped and the rates were fed with distilled water from the 31^{st} day. The rats in the normal group were given an equal volume of 0.5% CMC-Na solution by intragastric administration, while the rats in the high-dose almagate group and the low-dose almagate group were given a high dose of 0.6 g/kg and a low dose of 0.3 g/kg respectively by intragastric administration. The rats in the positive medicine group were given a dose of 10 mg/kg of sodium dimercaptosuccinate by intragastric administration, and all the intragastric subjects were given in equal volume twice a day for 30 days. On the 60^{th} day, blood samples were collected. Brain, liver and femur samples were collected and stored in liquid nitrogen for later use. After wet digestion, the blood samples and tissues of each group were tested for content of lead by inductively coupled plasma mass spectrometry (ICP-MS), and the lead-removing rates were counted. The results were shown in Tables 24 to 27.

All the data were processed by SPSS20.0 statistical software. Every group of measurement data was expressed by mean±relative standard deviation (mean±SD), and subjected to T test. p<0.05 was statistically significant.

### 6.5 Experimental Result

6.5.1 Effects of Administration of Almagate for Prevention on Lead in Blood, Lead in Brain and Lead in Bones in Chronic Rat Lead Poisoning Model

The effects of the administration of almagate for prevention on the lead in blood, the lead in brain and the lead in bones in the chronic rat lead poisoning model are shown in Tables 20 to 22.

TABLE 20
Effects of almagate on content of lead in blood in rats
with chronic lead poisoning (mean ± SD)

| | Number | | Content |
|---|---|---|---|
| | of animals | Dose | of lead |
| Group | (piece) | (g/kg) | (μg/g) |
| Normal group | 8 | — | 0.010 ± 0.02 |
| Model group | 8 | — | 0.056 ± 0.03^{##} |
| Low-dose | 8 | 0.3 | 0.047 ± 0.01 |
| almagate | | | |
| group | | | |
| High-dose | 8 | 0.6 | 0.035 ± 0.02* |
| almagate | | | |
| group | | | |

Note:
compared with the normal group: ^{##}p <0.01;
and compared with the model group: *p <0.05.

TABLE 21
Effects of almagate on content of lead in brain in rats with chronic lead
poisoning (mean ± SD)

| | Number of | Dose | Content of lead |
|---|---|---|---|
| Group | animals (piece) | (g/kg) | (μg/g) |
| Normal group | 8 | — | 0.023 ± 0.02 |
| Model group | 8 | — | 0.785 ± 0.12^{##} |
| Low-dose almagate | 8 | 0.3 | 0.716 ± 0.19 |
| group | | | |
| High-dose almagate | 8 | 0.6 | 0.682 ± 0.23* |
| group | | | |

Note:
compared with the normal group: ^{##}p < 0.01; and compared with the model group: *p < 0.05.

TABLE 22
Effects of almagate on femur content of lead in rats with chronic
lead poisoning (mean ± SD)

| | | | Content |
|---|---|---|---|
| | Number of | Dose | of lead |
| Group | animals (piece) | (g/kg) | (μg/g) |
| Normal group | 8 | — | 0.502 ± 0.56 |
| Model group | 8 | — | 60.431 ± 8.13^{##} |
| Low-dose almagate | 8 | 0.3 | 60.586 ± 10.25 |
| group | | | |
| High-dose almagate | 8 | 0.6 | 54.674 ± 4.96* |
| group | | | |

Note:
compared with the normal group: ^{##}p < 0.01; and compared with the model group: *p < 0.05.

The results are shown in Tables 21, 22 and 23. Compared with the normal group, the contents of the lead in blood, the lead in brain and the lead in bones in the model group and the almagate groups are significantly increased, indicating that modeling with this method is successful. By comparing the low-dose almagate group, the high-dose almagate group and the model group, the results show that the contents of the lead in blood, the lead in brain and the lead in bones of the high-dose almagate group are significantly lower than that of the model group, and are dose-dependent. Furthermore, the lead-removing rates of the almagate on various tissues of rats with chronic lead poisoning were calculated. The results are shown in Table 23. The effective rates of the almagate on the lead in brain at low and high doses are 8.79% and 13.12% respectively. The effective removing rates of the lead in blood at low and high doses are 16.07% and 37.50% respectively. The effective removing rates of the lead in bones at low and high doses are 0 and 9.53% respectively.

TABLE 23
Effective removing rates of almagate on lead in various tissues
of rats with chronic lead poisoning

| | Removing rate (%) | | |
|---|---|---|---|
| | Lead in | Lead in | Lead in |
| Group | brain | blood | bones |
| Low-dose group (0.3 g/kg) | 8.79 | 16.07 | 0 |
| High-dose group (0.6 g/kg) | 13.12 | 37.50 | 9.53 |

6.5.2 Effects of Administration of Almagate for Treatment on Lead in Blood, Lead in Brain and Lead in Bones in Chronic Rat Lead Poisoning Model

The effects of the administration of almagate for treatment on the lead in blood, the lead in brain and the lead in bones in the chronic rat lead poisoning model are shown in Tables 24 to 26.

TABLE 24
Effects of almagate on content of lead in blood in rats with chronic lead
poisoning (mean ± SD) on the 60^{th} day

| | Number of | Dose | Content of lead |
|---|---|---|---|
| Group | animals (piece) | (g/kg) | (μg/g) |
| Normal group | 8 | — | 0.016 ± 0.04 |
| Model group | 8 | — | 0.071 ± 0.02^{##} |
| Low-dose almagate | 8 | 0.3 | 0.082 ± 0.02 |
| group | | | |
| High-dose almagate | 8 | 0.6 | 0.055 ± 0.01* |
| group | | | |
| Sodium | 8 | 0.01 | 0.059 ± 0.01 |
| dimercaptosuccinate | | | |
| group | | | |

Note:
compared with the normal group: ^{##}p < 0.01; and compared with the model group: *p < 0.05.

TABLE 25
Effects of almagate on content of lead in brain in rats with chronic lead
poisoning (mean ± SD) on the 60^{th} day

| | Number of | Dose | Content of lead |
|---|---|---|---|
| Group | animals (piece) | (g/kg) | (μg/g) |
| Normal group | 8 | — | 0.019 ± 0.03 |
| Model group | 8 | — | 0.432 ± 0.36^{##} |
| Low-dose almagate | 8 | 0.3 | 0.297 ± 0.28 |
| group | | | |
| High-dose almagate | 8 | 0.6 | 0.149 ± 0.05* |
| group | | | |
| Sodium | 8 | 0.01 | 0.178 ± 0.04 |
| dimercaptosuccinate | | | |
| group | | | |

Note:
compared with the normal group: ^{##}p < 0.05; and compared with the model group: *p < 0.05.

TABLE 26
Effects of almagate on femur content of lead in rats with
chronic lead poisoning (mean ± SD) on the 60^{th} day

| | | | Content |
|---|---|---|---|
| | Number of | Dose | of lead |
| Group | animals (piece) | (g/kg) | (μg/g) |
| Normal group | 8 | — | 0.317 ± 0.296 |
| Model group | 8 | — | 23.573 ± 3.87^{##} |
| Low-dose almagate | 8 | 0.3 | 25.679 ± 4.26 |
| group | | | |
| High-dose almagate | 8 | 0.6 | 20.582 ± 2.93* |
| group | | | |
| Sodium | 8 | 0.01 | 28.075 ± 3.25 |
| dimercaptosuccinate | | | |
| group | | | |

Note:
compared with the normal group: ^{##}p < 0.01; and compared with the model group: *p < 0.05.

The results are shown in Tables 24, 25 and 26. Compared with the normal group, the contents of the lead in blood, the lead in brain and the lead in bones in the model group, the almagate groups and the positive medicine group (sodium dimercaptosuccinate group) are significantly increased, indicating that modeling with this method is successful. By comparing the model group, the low-dose almagate group and the high-dose almagate group, the results show that the contents of the lead in blood, the lead in brain and the lead in bones of the high-dose almagate group are significantly lower than that of the model group, and are dose-dependent. Furthermore, the lead-removing rates among the administration groups were analyzed. The details are shown in Table 27. The effective removing rate of the almagate administration group for treatment on the lead in brain is 22.54% at high dose, the effective removing rates of the almagate administration group for treatment on the lead in brain at low and high doses are 31.25% and 65.51% respectively, and the effective removing rate of the almagate administration group for treatment on the lead in bones at high dose is 12.69%.

TABLE 27
Effective removing rates of almagate on lead in various
tissues of rats with chronic lead poisoning

| | Removing rate (%) | | |
|---|---|---|---|
| | Positive | Low-dose almagate | High-dose almagate |
| Item | medicine group | group | group |
| Lead in | 16.90 | 0.00 | 22.54 |
| blood | | | |
| Lead in | 58.80 | 31.25 | 65.51 |
| brain | | | |
| Lead in | 0.00 | 0 | 12.69 |
| bones | | | |

### 6.6 Experimental Conclusion

According to the result judgment standard of “Method for the Assessment of Alleviating Lead Excretion Function” promulgated by China, compared with the model group, the lead in blood, the lead in brain and the lead in bones in the almagate groups are reduced significantly, and thus, it can be judged that the animal experimental results of the tested sample are positive. The efficacy results of the present invention show that the administration of almagate for prevention and treatment can effectively improve the contents of the lead in blood, the lead in brain and the lead in bones in the rats with chronic lead poisoning.

In conclusion, the almagate has the pharmacological action of removing lead, and has good preventive and therapeutic effects when being used as a lead-removing agent.

### Embodiment 7 Synthesis of Mg₅ZnAl₂(OH)₁₆CO₃.4H₂O and In Vitro Study on Adsorption of Lead

7.1 Synthesis of Mg₅ZnAl₂(OH)₁₆CO₃.4H₂O

7.1.1 Medicine and Reagent

Al2(SO₄)₃.18H₂O (Sinopharm), MgSO₄.7H₂O (Sinopharm), ZnSO₄.7H₂O (Sinopharm), Na₂CO₃ (Sinopharm), and NaOH (Sinopharm).

7.1.2 Synthesis Method of Mg₅ZnAl₂(OH)₁₆CO₃.4H₂O

33.8 g of Al₂(SO₄)₃.18H₂O, 61.6 g of MgSO₄.7H₂O and 14.4 g of ZnSO₄.7H₂O were weighed and dissolved with deionized water to prepare 250 ml of solution. 21.5 g of Na₂CO₃ and 27.5 g of NaOH were weighed and dissolved with deionized water to prepare 250 ml of solution.

A 1000 ml four-necked round bottom flask was taken, added with 100 ml of deionized water, heated to 63° C. to 67° C., dropwise added with the above two solutions while stirring, then the pH value of the reaction system was controlled to be between 9.8 and 10.0, and the solutions were dropwise added completely in half an hour. The temperature was raised to 90° C. to 95° C., and then maintained for 24 hours. Then, the reaction system was cooled to 65° C., filtered, washed with deionized water to the pH being 8.5 to 9.5, dried, crushed and sieved with a 80-mesh sieve to obtain 32.0 g of white product.

According to the chemical industry standard HG/T 3820-2013 of the People's Republic of China, the product was analyzed. The analysis results are that: the content of magnesium oxide is 31.1%, the content of aluminum oxide is 15.8%, and the content of zinc oxide is 12.7%. 7.2 In vitro study on adsorption of lead by Mg₅ZnAl₂(OH)₁₆CO₃.4H₂O

### 7.2.1 Medicine and Reagent

Mg₅ZnAl₂(OH)₁₆CO₃.4H₂O (self-prepared); lead acetate (Sinopharm); potassium dihydrogen phosphate (Sinopharm); and sodium hydroxide (Sinopharm).

### 7.2.2 Reagent Preparation

Buffer solution: 6.8 g of potassium dihydrogen phosphate was weighed and added with 500 mL of water. The pH was adjusted to 6.8 with 0.4% sodium hydroxide solution; and then water was added to a constant volume of 1,000 mL.

Lead solution: 0.1599 g of lead nitrate was weighed and placed in a 1000 mL volumetric flask, added 50 mL of buffer solution to dissolve, and diluted to scale with the buffer solution. Every milliliter contained 100 mg of Pb.

7.2.3 In Vitro Study on Adsorption of Lead by Mg₅ZnAl₂(OH)₁₆CO₃.4H₂O

10 mL of lead solution (100 μg/mL) was weighed, placed in a 100 mL volumetric flask, added with buffer solution and diluted to scale, and then shaken evenly (each milliliter contained 10 μg of Pb). Then, the mixture was transferred to a container, and stirred at a constant temperature of 37° C. under airtight conditions for 30 minutes.

10 mg of sample (Mg₅ZnAl₂(OH)₁₆CO₃.4H₂O) was weighed and added into the above-mentioned lead solution (10 μg/mL) to form a mixed system, and stirred at 37° C. under airtight conditions for 2 hours. The samples were taken and filtered at the 15^{th} minute, the 30^{th} minute, the 45^{th} minute, the 60^{th} minute and 120^{th} minute respectively. The pH value of the adsorbed solution was determined and the concentration of the lead irons was determined. As a reference, the lead solution stock solution (10 μg/mL) without the sample was filtered and the lead ion concentration therein was detected. Lead was detected by plasma atomic emission spectrometry. The detection results are shown in Table 28.

TABLE 28
In vitro study result on adsorption of lead by
Mg₅ZnAl₂(OH)₁₆CO₃•4H₂O

| | | | | | | |
|---|---|---|---|---|---|---|
| Time (min) | — | 15 | 30 | 45 | 60 | 120 |
| Lead concentration | 9.94 | 1.76 | 1.49 | 1.18 | 0.97 | 0.63 |
| (μg/mL) | | | | | | |
| Adsorption rate | — | 82.4 | 85.1 | 88.2 | 90.3 | 93.7 |
| (%) | | | | | | |

It can be seen from Table 28 that the adsorption rate of Mg₅ZnAl₂(OH)₁₆CO₃.4H₂O on the lead is higher than 80% at the 15th minute, and the adsorption effect is obvious.

### Embodiment 8 Study on the Prevention and Treatment of Chronic Lead Poisoning in Rats with Mg₅ZnAl₂(OH)₁₆CO₃.4H₂O

### 8.1 Medicine and Reagent

Mg₅ZnAl₂(OH)₁₆CO₃.4H₂O (synthesized by the method of Embodiment 7); lead acetate (Sinopharm); perchloric acid (Sinopharm); and sodium dimercaptosuccinate (Shanghai New Asiatic Pharmaceutical).

### 8.2 Experimental Animal

Male SD rats, weighing 130 g to 150 g, provided by the Animal Center of Shanghai Xipuer-Bikai Laboratory Animal Co., Ltd. (Certificate No.: SCXK 2008-0016).

### 8.3 Main Instrument

Assay balance (METTLER TOLEDO, Swiss, and model: PL 601-L); rat scale (Nanjing Emmanuel Instrument Equipment Co., Ltd.); and series 7700 ICP-MS (Agilent, USA).

### 8.4 Experimental Method

(1) Chronic Rat Lead Poisoning Model and Administration for Prevention

32 male SD rats were randomly divided into four groups including a normal group, a model group, a high-dose Mg₅ZnAl₂(OH)₁₆CO₃.4H₂O group and a low-dose Mg₅ZnAl₂(OH)₁₆CO₃.4H₂O group according to their body weights, and each group included 8 rats. Except the rats in the normal group were given ultrapure water, the rats in the model group, the positive medicine group, and the Mg₅ZnAl₂(OH)₁₆CO₃.4H₂O groups of each dose drank 0.5% lead acetate ultrapure water solution for 30 days, resulting in chronic lead-exposed models. From the 4th day of modeling, the rats in the high-dose Mg₅ZnAl₂(OH)₁₆CO₃.4H₂O group and the low-dose Mg₅ZnAl₂(OH)₁₆CO₃.4H₂O group were given a high dose of 0.6 g/kg and a low dose of 0.3 g/kg, respectively (the Mg₅ZnAl₂(OH)₁₆CO₃.4H₂O and deionized water were mixed to make suspension, which was given by intragastric administration with equal volume). The rats in the normal group were given equal volume of 0.5% CMC-Na solution by intragastric administration. All the subjects subjected to intragastric administration were given in equal volume twice a day for 30 days. After 30 days, blood samples were collected from aorta abdominalis, and the rats were sacrificed under excessive anesthesia. Brain, liver and femur samples were collected and stored in liquid nitrogen for later use. After wet digestion, the blood samples and tissues of each group were tested for content of lead by inductively coupled plasma mass spectrometry (ICP-MS), and the lead-removing rates were counted. The results were shown in Tables 29 to 32.

(2) Chronic Rat Lead Poisoning Model and Administration for Treatment

40 male SD rats were randomly divided into five groups including a normal group, a model group, a positive medicine group (sodium dimercaptosuccinate group), high-dose MgsZnAl₂(OH)₁₆CO₃.4H₂O group and a low-dose Mg₅ZnAl₂(OH)₁₆CO₃.4H₂O group according to their body weights, and each group included 8 rats. Except the rats in the normal group were given ultrapure water, the rats in the model group, the positive medicine group, and the Mg₅ZnAl₂(OH)₁₆CO₃.4H₂O groups of each dose drank 0.5% lead acetate ultrapure water solution for 30 days, resulting in chronic lead-exposed models. After the model was successfully established, the lead was stopped and the rates were fed with distilled water from the 31^{st} day. The rats in the normal group were given an equal volume of 0.5% CMC-Na solution by intragastric administration, while the rats in the high-dose Mg₅ZnAl₂(OH)₁₆CO₃.4H₂O group and the low-dose Mg₅ZnAl₂(OH)₁₆CO₃.4H₂O group were given a high dose of 0.6 g/kg and a low dose of 0.3 g/kg respectively by intragastric administration. The rats in the positive medicine group were given a dose of 10 mg/kg of sodium dimercaptosuccinate by intragastric administration, and all the intragastric subjects were given in equal volume twice a day for 30 days. On the 60th day, blood samples were collected. Brain, liver and femur samples were collected and stored in liquid nitrogen for later use. After wet digestion, the blood samples and tissues of each group were tested for content of lead by inductively coupled plasma mass spectrometry (ICP-MS), and the lead-removing rates were counted. The results were shown in Tables 33 to 36.

All the data were processed by SPSS20.0 statistical software. Every group of measurement data was expressed by mean±relative standard deviation (mean±SD), and subjected to T test. p<0.05 was statistically significant.

### 8.5 Experimental Result

8.5.1 Effects of administration of Mg₅ZnAl₂(OH)₁₆CO₃.4H₂O for prevention on lead in blood, lead in brain and lead in bones in chronic rat lead poisoning model

The effects of the administration of Mg₅ZnAl₂(OH)₁₆CO₃.4H₂O for prevention on the lead in blood, the lead in brain and the lead in bones in the chronic rat lead poisoning model are shown in Tables 29 to 31.

TABLE 29
Effects of Mg₅ZnAl₂(OH)₁₆CO₃•4H₂O on content of lead in blood in
rats with chronic lead poisoning (mean ± SD)

| | | | Content |
|---|---|---|---|
| | Number of | Dose | of lead |
| Group | animals (piece) | (g/kg) | (μg/g) |
| Normal group | 8 | — | 0.028 ± 0.02 |
| Model group | 8 | — | 0.085 ± 0.03^{##} |
| Low-dose | 8 | 0.3 | 0.072 ± 0.05 |
| Mg₅ZnAl₂(OH)₁₆CO₃•4H₂O | | | |
| group | | | |
| High-dose | 8 | 0.6 | 0.056 ± 0.01* |
| Mg₅ZnAl₂(OH)₁₆CO₃•4H₂O | | | |
| group | | | |

Note:
compared with the normal group: ^{##}p < 0.01; and compared with the model group: *p < 0.05.

TABLE 30
Effects of Mg₅ZnAl₂(OH)₁₆CO₃•4H₂O on content of lead in
brain in rats with chronic lead poisoning (mean ± SD)

| | Number of | Dose | Content of lead |
|---|---|---|---|
| Group | animals (piece) | (g/kg) | (μg/g) |
| Normal group | 8 | — | 0.058 ± 0.02 |
| Model group | 8 | — | 0.617 ± 0.08^{##} |
| Low-dose | 8 | 0.3 | 0.526 ± 0.11 |
| Mg₅ZnAl₂(OH)₁₆CO₃•4H₂O | | | |
| group | | | |
| High-dose | 8 | 0.6 | 0.488 ± 0.17* |
| Mg₅ZnAl₂(OH)₁₆CO₃•4H₂O | | | |
| group | | | |

Note:
compared with the normal group: ^{##}p < 0.01; and compared with the model group: *p < 0.05.

TABLE 31
Effects of Mg₅ZnAl₂(OH)₁₆CO₃•4H₂O on femur content of
lead in rats with chronic lead poisoning (mean ± SD)

| | Number of | Dose | Content of lead |
|---|---|---|---|
| Group | animals (piece) | (g/kg) | (μg/g) |
| Normal group | 8 | — | 0.337 ± 0.31 |
| Model group | 8 | — | 45.163 ± 6.24^{##} |
| Low-dose | 8 | 0.3 | 47.798 ± 9.72 |
| Mg₅ZnAl₂(OH)₁₆CO₃•4H₂O | | | |
| group | | | |
| High-dose | 8 | 0.6 | 40.367 ± 4.01* |
| Mg₅ZnAl₂(OH)₁₆CO₃•4H₂O | | | |
| group | | | |

Note:
compared with the normal group: ^{##}p < 0.01; and compared with the model group: *p < 0.05.

The results are shown in Tables 29, 30 and 31. Compared with the normal group, the contents of the lead in blood, the lead in brain and the lead in bones in the model group and the Mg₅ZnAl₂(OH)₁₆CO₃.4H₂O groups are significantly increased, indicating that modeling with this method is successful. By comparing the low-dose Mg₅ZnAl₂(OH)₁₆CO₃.4H₂O group, the high-dose Mg₅ZnAl₂(OH)₁₆CO₃.4H₂O group and the model group, the results show that the contents of the lead in blood, the lead in brain and the lead in bones of the high-dose Mg₅ZnAl₂(OH)₁₆CO₃.4H₂O group are significantly lower than that of the model group, and are dose-dependent. Furthermore, the lead-removing rates of the Mg₅ZnAl₂(OH)₁₆CO₃.4H₂O on various tissues of rats with chronic lead poisoning were calculated. The results are shown in Table 32. The effective rates of the Mg₅ZnAl₂(OH)₁₆CO₃.4H₂O on the lead in brain at low and high doses are 14.75% and 22.91% respectively. The effective removing rates of the lead in blood at low and high doses are 15.29% and 34.12% respectively. The effective removing rates of the lead in bones at low and high doses are 0 and 10.62% respectively.

TABLE 32
Effective removing rates of Mg₅ZnAl₂(OH)₁₆CO₃•4H₂O
on lead in various tissues of rats with chronic lead poisoning

| | Removing rate (%) | | |
|---|---|---|---|
| | Lead in | Lead in | Lead in |
| Group | brain | blood | bones |
| Low-dose group (0.3 g/kg) | 14.75 | 15.29 | 0 |
| High-dose group (0.6 g/kg) | 22.91 | 34.12 | 10.62 |

8.5.2 Effects of Administration of Mg₅ZnAl₂(OH)₁₆CO₃.4H₂O for Treatment on Lead in Blood, Lead in Brain and Lead in Bones in Chronic Rat Lead Poisoning Model

The effects of the administration of Mg₅ZnAl₂(OH)₁₆CO₃.4H₂O for treatment on the lead in blood, the lead in brain and the lead in bones in the chronic rat lead poisoning model are shown in Tables 33 to 35.

TABLE 33
Effects of Mg₅ZnAl₂(OH)₁₆CO₃•4H₂O on content of lead in blood in
rats with chronic lead poisoning (mean ± SD) on the 60^{th} day

| | Number of | | Content |
|---|---|---|---|
| | animals | Dose | of lead |
| Group | (piece) | (g/kg) | (μg/g) |
| Normal group | 8 | — | 0.015 ± 0.03 |
| Model group | 8 | — | 0.083 ± 0.04^{##} |
| Low-dose | 8 | 0.3 | 0.093 ± 0.02 |
| Mg₅ZnAl₂(OH)₁₆CO₃•4H₂O group | | | |
| High-dose | 8 | 0.6 | 0.069 ± 0.01* |
| Mg₅ZnAl₂(OH)₁₆CO₃•4H₂O group | | | |
| Sodium dimercaptosuccinate group | 8 | 0.01 | 0.073 ± 0.01 |

Note:
compared with the normal group: ^{##}p < 0.01; and compared with the model group: *p < 0.05.

TABLE 34
Effects of Mg₅ZnAl₂(OH)₁₆CO₃•4H₂O on content of lead in
brain in rats with chronic lead poisoning (mean ± SD) on the 60^{th} day

| | | | Content |
|---|---|---|---|
| | Number of | Dose | of lead |
| Group | animals (piece) | (g/kg) | (μg/g) |
| Normal group | 8 | — | 0.031 ± 0.04 |
| Model group | 8 | — | 0.498 ± 0.42^{#} |
| Low-dose | 8 | 0.3 | 0.326 ± 0.12 |
| Mg₅ZnAl₂(OH)₁₆CO₃•4H₂O | | | |
| group | | | |
| High-dose | 8 | 0.6 | 0.152 ± 0.08* |
| Mg₅ZnAl₂(OH)₁₆CO₃•4H₂O | | | |
| group | | | |
| Sodium | 8 | 0.01 | 0.165 ± 0.05 |
| dimercaptosuccinate group | | | |

Note:
compared with the normal group: ^{#}p < 0.05; and compared with the model group: *p < 0.05.

TABLE 35
Effects of Mg₅ZnAl₂(OH)₁₆CO₃•4H₂O on femur content of lead
in rats with chronic lead poisoning (mean ± SD) on the 60^{th} day

| | | | Content |
|---|---|---|---|
| | Number of | Dose | of lead |
| Group | animals (piece) | (g/kg) | (μg/g) |
| Normal group | 8 | — | 0.481 ± 0.51 |
| Model group | 8 | — | 28.376 ± 4.93^{#} |
| Low-dose | 8 | 0.3 | 28.562 ± 5.27 |
| Mg₅ZnAl₂(OH)₁₆CO₃•4H₂O | | | |
| group | | | |
| High-dose | 8 | 0.6 | 22.793 ± 4.52* |
| Mg₅ZnAl₂(OH)₁₆CO₃•4H₂O | | | |
| group | | | |
| Sodium | 8 | 0.01 | 33.478 ± 4.86 |
| dimercaptosuccinate group | | | |

Note:
compared with the normal group: ^{#}p < 0.01; and compared with the model group: *p < 0.05.

The results are shown in Tables 33, 34 and 35. Compared with the normal group, the contents of the lead in blood, the lead in brain and the lead in bones in the model group, the Mg₅ZnAl₂(OH)₁₆CO₃.4H₂O groups and the positive medicine group (sodium dimercaptosuccinate group) are significantly increased, indicating that modeling with this method is successful. By comparing the model group, the low-dose Mg₅ZnAl₂(OH)₁₆CO₃.4H₂O group and the high-dose Mg₅ZnAl₂(OH)₁₆CO₃.4H₂O group, the results show that the contents of lead in blood, the lead in brain and the lead in bones of the high-dose Mg₅ZnAl₂(OH)₁₆CO₃.4H₂O group are significantly lower than that of the model group, and are dose-dependent. Furthermore, the lead-removing rates among the administration groups were analyzed. The details are shown in Table 36. The effective removing rate of the Mg₅ZnAl₂(OH)₁₆CO₃.4H₂O administration group for treatment on the lead in brain is 16.87% at high dose, the effective removing rates of the Mg₅ZnAl₂(OH)₁₆CO₃.4H₂O administration group for treatment on the lead in brain at low and high doses are 34.54% and 69.48% respectively, and the effective removing rate of the Mg₅ZnAl₂(OH)₁₆CO₃.4H₂O administration group for treatment on the lead in bones at high dose is 19.72%.

TABLE 36
Effective removing rates of Mg₅ZnAl₂(OH)₁₆CO₃•4H₂O on lead
in various tissues of rats with chronic lead poisoning

| | Removing rate (%) | | |
|---|---|---|---|
| | Pos- | | |
| | itive | | |
| | med- | Low-dose | High-dose |
| | icine | Mg₅ZnAl₂(OH)₁₆CO₃•4H₂O | Mg₅ZnAl₂(OH)₁₆CO₃•4H₂O |
| Item | group | group | group |
| Lead | 12.05 | 0.00 | 16.87 |
| in | | | |
| blood | | | |
| Lead | 66.87 | 34.54 | 69.48 |
| in | | | |
| brain | | | |
| Lead | 0.00 | 0.00 | 19.72 |
| in | | | |
| bones | | | |

### 8.6 Experimental Conclusion

According to the result judgment standard of “Method for the Assessment of Alleviating Lead Excretion Function” promulgated by China, compared with the model group, the lead in blood, the lead in brain and the lead in bones in the Mg₅ZnAl₂(OH)₁₆CO₃.4H₂O groups are reduced significantly, and thus, it can be judged that the animal experimental results of the tested sample are positive. The efficacy results of the present invention show that the administration of Mg₅ZnAl₂(OH)₁₆CO₃.4H₂O for prevention and treatment can effectively improve the contents of the lead in blood, the lead in brain and the lead in bones in the rats with chronic lead poisoning.

In conclusion, the Mg₅ZnAl₂(OH)₁₆CO₃.4H₂O has the pharmacological action of removing lead, and has good preventive and therapeutic effects when being used as a lead-removing agent.

What is claimed is:

1. A use of anionic clay in preparing a lead-removing medicine.

2. The use according to claim 1, wherein a composition or compound medicine composed of the anionic clay and another pharmaceutically acceptable therapeutic agent is used for preparing a lead-removing medicine.

3. The use according to claim 1, wherein a pharmaceutically acceptable adjuvant is added into the anionic clay to prepare a capsule, a tablet, a powder, a granule, a suspension or a suspensoid.

4. The use according to claim 2, wherein a pharmaceutically acceptable adjuvant is added into the composition or compound medicine to prepare a capsule, a tablet, a powder, a granule, a suspension or a suspensoid.

5. The use according to claim 1, wherein the lead-removing medicine is capable of removing lead in blood, lead in brain or lead in bones.
